(19) United States
(12) Patent Application Publication
(10) Pub. No.: US 2003/0023180 A1
(43) Pub. Date: Jan. 30, 2003

(54) Respiratory analyzer and method for measuring changes in concentration of a gas component of a breathing gas mixture

(76) Inventor: James R. Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/162,371

(22) Filed: Jun. 4, 2002

Related U.S. Application Data
(60) Provisional application No. 60/308,043, filed on Jul. 26, 2001.

(51) Int. Cl. A61B 5/08

(52) U.S. Cl. 600/531

(57) ABSTRACT

A method and apparatus for measuring changes in the concentration of a particular gas component of a breathing gas mixture after inhalation and exhalation by means of a degradable sensor. This is done by exposing, during inhalation, the sensor to a breathing gas mixture of known concentration to produce an inhalation output corresponding to the measured concentration of the gas component in the inhalation. From the known concentration of the gas component in the breathing gas mixture and the measured concentration in the inhalation output, a degradation parameter is determined representing the instantaneous degradation condition of the sensor. The sensor is exposed to the breathing gas mixture during exhalation to produce an exhalation output corresponding to the measured concentration of the gas component in the exhalation; the exhalation output of the sensor is modified according to the determined degradation parameter; and the change in concentration is computed from the modified exhalation output and the measured inhalation output.

### RELATED APPLICATION

The present application includes subject matter described in Provisional Application Serial No. 60/308,043 filed Jul. 26, 2001, the complete disclosure of which is incorporated herein by reference, and claims the priority date thereof.

### FIELD OF THE INVENTION

The present invention relates to a method, and also to a respiratory analyzer, for measuring changes in the concentration of a particular gas component of a breathing gas mixture after inhalation and exhalation. The invention is particularly useful in a method, and also in an indirect calorimeter, for measuring the metabolic rate of the individual, e.g., in a diet or weight-control program, and is therefore described below with respect to such application.

### BACKGROUND OF THE INVENTION

A respiratory analyzer includes a flow channel through which a subject breathes a breathing gas mixture, typically atmospheric air, and one or more sensors which sense and measure one or more gas components of the breathing gas mixture. Such analyzers are widely used for measuring metabolism and related respiratory parameters, by indirect calorimetry in a diet or weight-control program.

The term “respiratory analyzer”, as used herein, refers to any device used to study the breath of a person, such as an indirect calorimeter (including oxygen consumption meters and carbon dioxide production meters), breath diagnostic systems, ventilator control systems, spirometers, nitric oxide meters, and other devices. A particularly important application of such analyzers is to measure oxygen concentration, which may be done in a variety of ways. The preferred embodiments of the invention described below utilize oxygen sensors of the fluorescent-type, where molecular oxygen is the quenching species. Accordingly, the invention is described below particularly with respect to such sensors, but it will be appreciated that the invention can be used with gas component sensors other than the fluorescent-type.

The fluorescent-type sensor provides a fluorescence signal correlated with the partial pressure of the gas component to be sensed (e.g., oxygen) in the respired gases. Consider, for example, inhaled gas having an inhaled oxygen partial pressure Pₗ, and exhaled gas having an exhaled oxygen partial pressure Pₑ. It will also be assumed that the fluorescent signal is related to a fluorescent intensity of a sensing channel, although fluorescence decay and attack times can also be used to determine oxygen partial pressures in a manner known to the art.

The fluorescence sensor provides a fluorescent intensity signal Fᵢ during inhalation, and a fluorescent intensity signal Fₑ during exhalation. The difference in fluorescence signals (ΔF), which equals Fₑ−Fᵢ, is correlated with the difference in oxygen partial pressures (ΔP), which equals Pᵢ−Pₑ.

An oxygen sensor can be calibrated so that Fₑ and Fᵢ have a known correlation with Pₑ and Pᵢ, using a numerical or analytical relationship or some combination thereof. The term “fluorescence-oxygen relationship” will be used to refer to the fluorescence intensity/oxygen partial pressure relationship and the fluorescence decay time (or excited state lifetime)/oxygen partial pressure relationship for a fluorescent material. The term “sensor response function” will be used in a more general sense, to refer to a fluorescence-oxygen relationship, but also to other sensor responses to analytes. The fluorescence-oxygen relationship determined for a new sensor (i.e. before degradation) will be termed the “initial fluorescence-oxygen relationship”.

U.S. Pat. Nos. 5,917,605, 5,910,661, 5,894,351, and 5,517,313, the contents of which are incorporated herein by reference, describe fluorescence sensors for various analytes, including oxygen and glucose in a fluid. An oxygen sensing film can include, for example, a ruthenium (II) complex irradiated by an excitation radiation source so as to induce an orange-red fluorescence. The fluorescence is quenched by the presence of molecular oxygen, reducing the fluorescence intensity in a manner which can be correlated with the partial pressure of oxygen.

Sensors can include a sensing channel and a reference channel. The sensing channel can be, for example, a fluorescent film exposed to the fluid and providing a fluorescence signal correlated with the partial pressure of the fluid component of interest. The reference channel can be a similar fluorescent film exposed to the same environmental conditions, such as temperature and excitation radiation intensity, but not exposed to the fluid. A fluorescence intensity ratio (between the sensing channel fluorescence intensity and the reference channel fluorescence intensity) will be correlated with the partial pressure of the fluid component, but not correlated with environmental factors common to both channels, such as the instantaneous temperatures or excitation radiation intensity. The use of a reference channel therefore provides compensation for the instantaneous temperature and/or other environmental influences.

Fluorescence quenching and fluorescence lifetime of fluorescence quenching sensors follow the Stern-Vollmer equation, for example, as discussed in U.S. Pat. No. 6,074,607 to Slovacek et al. and U.S. Pat. No. 5,518,694 to Bentsen, the contents of which are incorporated herein by reference. The equation is written in the form:

F/F₀=t/t₀=1+k_{q}t₀[Q]

where F₀ is the fluorescence intensity for zero concentration of the quencher, F is the fluorescence intensity in the presence of the quencher, τ₀ is the excited state lifetime for zero concentration of the quencher, τ is the excited state lifetime in presence of the quencher, k_{q} is the bimolecular fluorescence quenching rate constant, and [Q] is the concentration of the quencher in the fluorescent sensor film.

This equation is often written as:

F/F₀=t/t₀=1+K_{SV}P_{Q}

where k_{SV} is the Stem-Vollmer coefficient, and P_{Q} is the partial pressure of the quencher.

Molecular oxygen (O₂) is known to quench the fluorescence of various fluorescent films. The Stem-Vollmer coefficients account for the solubility of oxygen, or other analyte, in the fluorescent element. In the description below, the representation P is used to refer to the partial pressure of analytes, such as oxygen or carbon dioxide in fluid flow.

Fluorescence decay times, or excited state lifetimes, can be measured using excitation source modulation techniques, for example as described by U.S. Pat. No. 5,518,694 to Bentsen. Fluorescent intensities are conventionally measured using a photodetector receiving the fluorescence. An optical filter can be used to prevent excitation radiation from reaching the photodetector.

U.S. patent application Ser. No. 09/630,398 describes an indirect calorimeter, particularly useful for determining metabolic rates in a dietary management and/or weight control program, including a flow pathway, a flow sensor, and an oxygen sensor. The integration of flow rate and oxygen partial pressure gives oxygen volumes in respired gases. The oxygen consumed by a person is the difference between inhaled and exhaled oxygen volumes (corrected to standard conditions). A metabolic rate may be determined from the consumed oxygen volume. In other embodiments, a carbon dioxide sensor or capnometer can be used instead of or in addition to the oxygen sensor to determine metabolic rate.

U.S. patent application Ser. No. 09/630,398 also describes an oxygen sensor having a sensing channel and a reference channel. The sensing channel includes a fluorescent film exposed to changes in oxygen partial pressure in respired gases passing through the indirect calorimeter, and provides a fluorescence signal correlated with the oxygen partial pressure in the respired gases. The reference channel is part of the same sensor package and is similarly affected by ambient conditions such as temperature. However, the reference channel is not exposed to the oxygen in the respired gas. Therefore, the ratio of the sensing channel fluorescence signal to the reference channel fluorescence signal is correlated with oxygen partial pressure in the respired gases, but not with ambient conditions, as the effects of ambient conditions on sensing and reference channels (ideally) cancel out. The use of a reference channel, however, adds to the cost and complexity of the sensor.

In embodiments of an indirect calorimeter described in U.S. patent application Ser. No. 09/630,398, a single-point recalibration technique is used to compensate for the temperature changes of the oxygen sensor. Single point recalibration of an oxygen sensor is also described in the above-cited Bentsen U.S. Pat. No. 5,518,694.

Over time, the fluorescence intensity from a fluorescent material can drop because of degradation. Degradation may be due to photochemical processes, contamination, decomposition, oxidation, and the like, of the fluorescent material or the supporting matrix. In a sensor having a sensing channel and a reference channel, degradation of the reference channel does not follow the same time dependence as the degradation of the sensing channel because the reference sensor is not exposed to the flowing fluid. Thus, the sensing channel generally degrades faster than the reference channel, for example due to exposure to contaminants, oxidants, and the like in the fluid to which the sensing sensor is exposed. The correlation between the fluorescent intensity ratio and fluid component partial pressure will, therefore, change according to the to different degradation rates of the sensing and reference channels. Such degradation of the sensor therefore requires frequent recalibration, which is a time-consuming and frequently not convenient.

It would, therefore, be clearly advantageous to provide a method and apparatus which automatically corrects for the degradation condition of the sensor.

Further, a reference channel provides no analytical information regarding the fluid, such as respired gases. It may be advantageous in some applications to use an additional sensing film in place of the reference channel to provide additional information, such as the presence and partial pressure of other gas components. It may also be advantageous to omit the reference channel in order to provide a sensor with lower cost, complexity, and power consumption. While conventional sensors exist in which only a sensing channel is present, such sensors are sensitive to environmental conditions such as temperature. It would be clearly advantageous to obviate the need for the reference channel while still providing oxygen (or other gas component) partial pressure measurements of similar or greater accuracy as conventional sensors having a reference channel.

### OBJECTS AND BRIEF SUMMARY OF THE PRESENT INVENTION

One object of the present invention is to provide a method for measuring changes in the concentration of a particular gas component of a breathing gas mixture which method automatically corrects for the degradation condition of the sensor. Another object of the invention is to provide a method which does not require the inclusion of a reference channel, although such a channel may be included if desired for greater accuracy. Further objects of the invention are to provide a respiratory analyzer and an indirect calorimeter operating according to the novel method and providing the foregoing advantages.

According to one aspect of the present invention, there is provided a method of measuring changes in the concentration of a particular gas component of a breathing gas mixture after inhalation and exhalation of the breathing gas mixture, which breathing gas mixture has a known concentration of the particular gas component before inhalation thereof. This is done by exposing a sensor for the gas component to the breathing gas mixture during inhalation to produce an inhalation output from the sensor corresponding to the measured concentration of the gas component in the inhalation. The sensor used is one which is degradable by usage. From the known concentration of the gas component in the breathing gas mixture and the measured concentration in the inhalation output, a degradation parameter is determined representing the instantaneous degradation condition of the sensor. The sensor is exposed to the breathing gas mixture during exhalation to produce an exhalation output from the sensor corresponding to the measured concentration of the gas component in the exhalation. The exhalation output from the sensor is modified according to the determined degradation parameter; and the change in concentration is computed from the modified exhalation output and the measured inhalation output.

According to one described preferred embodiment, the gas component is oxygen, and the change in concentration is computed by subtracting the modified exhalation output from the measured inhalation output. According to another described embodiment, the gas component is carbon dioxide (alone, or together with oxygen); and the change in concentration is computed by subtracting the measured inhalation carbon dioxide output from the modified exhalation carbon dioxide output. It will be appreciated that other embodiments could be provided wherein changes in concentration of other gas components, such as nitric oxides, are measured.

In all the preferred embodiments described below, the degradable sensor is a fluorescent-type sensor which produces an output corresponding to the partial pressure of the gas component of interest. In the described preferred embodiments, the breathing gas mixture inhaled and exhaled is atmospheric air having a known concentration in its dry state, i.e. after subtracting the humidity content. Therefore, in the described preferred embodiment, the instantaneous humidity concentration of the atmospheric air is determined and used to correct the known concentration of the gas component in the atmospheric air for determining the degradation parameter.

In the preferred embodiment of the invention described below, a reference sensor for the gas component, while exposed to the instantaneous temperature as the first-mentioned sensor but not exposed to the breathing gas mixture, is utilized during the inhalation and exhalation to produce reference outputs corresponding to the instantaneous temperature, which reference outputs are used to correct the inhalation and exhalation outputs of the first-mentioned sensor for the instantaneous temperature during the inhalation and exhalation.

According to another aspect of the present invention, there is provided a method of measuring the metabolic rate of a subject comprising: measuring, in accordance with the above defined method, changes in the concentration of a particular gas component of a breathing gas mixture after inhalation and exhalation by the subject; measuring the volume of the breathing gas mixture inhaled and exhaled by the subject; and multiplying the measured volume by the measured changes in the concentration of the gas component to provide a measurement of the metabolic rate of the subject.

According to a further aspect of the present invention, there is provided a respiratory analyzer for measuring changes in the concentration of a particular gas component of a breathing gas mixture after inhalation and exhalation of the breathing gas mixture, which breathing gas mixture has a known concentration of the gas component before inhalation thereof. The analyzer comprises: a common flow path for the breathing gas mixture during inhalations and exhalations thereof; a sensor exposed to the breathing gas mixture in the flow path to produce an inhalation output from the sensor and an exhalation output from the sensor corresponding to the measured concentration of the gas component in the inhalations and exhalations, respectively; and a processor for: determining, from the known concentration of the gas component in the breathing gas mixture and the measured concentration in the inhalation output from the sensor, a degradation parameter representing the instantaneous degradation condition of the sensor; modifying the exhalation output of the sensor according to the determined degradation parameter; and computing the change in concentration from the modified exhalation output and the measured inhalation output from the sensor.

According to a still further aspect of the present invention, there is provided an indirect calorimeter for measuring the metabolic rate of a subject, comprising a respiratory analyzer as set forth above for measuring changes in the concentration of a particular gas component of a breathing gas mixture after inhalation and exhalation thereof; and an apparatus for measuring the volume of the breathing gas mixture inhaled and exhaled by the subject; the processor also multiplying the measured volume by the measured changes in the concentration of the gas component to provide measurement of the metabolic rate of the subject. Such a calorimeter is particularly useful in a diet or weight-control program.

Further features and advantages of the invention will be apparent from the description below.

### BRIEF DESCRIPTION OF THE DRAWINGS

The invention is herein described, somewhat diagrammatically and by way of example only, with reference to the accompanying drawings, wherein:

FIG. 1 is a diagram illustrating the relationship between a fluorescence intensity signal (F) and the oxygen partial pressure (P) for a fluorescent film-type sensor exposed to different partial pressures of a particular gas of interest, such as oxygen, in a mixture of gases, such as a breathing gas mixture;

FIG. 2 is a diagram illustrating the manner in which an unknown gas partial pressure (P₂) can be determined from the relationship F₁, P₁ illustrated in FIG. 1;

FIG. 3 illustrates the relationship of FIG. 1 in a non-degraded sensor and in a degraded sensor, respectively;

FIG. 4 illustrates the relationship of FIG. 2 in a non-degraded sensor and in a degraded sensor, respectively;

FIG. 5 illustrates a typical manner in which the degradation parameter (D) varies with ΔF for a given P₁−P₂, or ΔP;

FIG. 6 is a block diagram illustrating an indirect calorimeter constructed in accordance with the present invention; and

FIG. 7 is a flow chart illustrating the operation of the indirect calorimeter of FIG. 6.

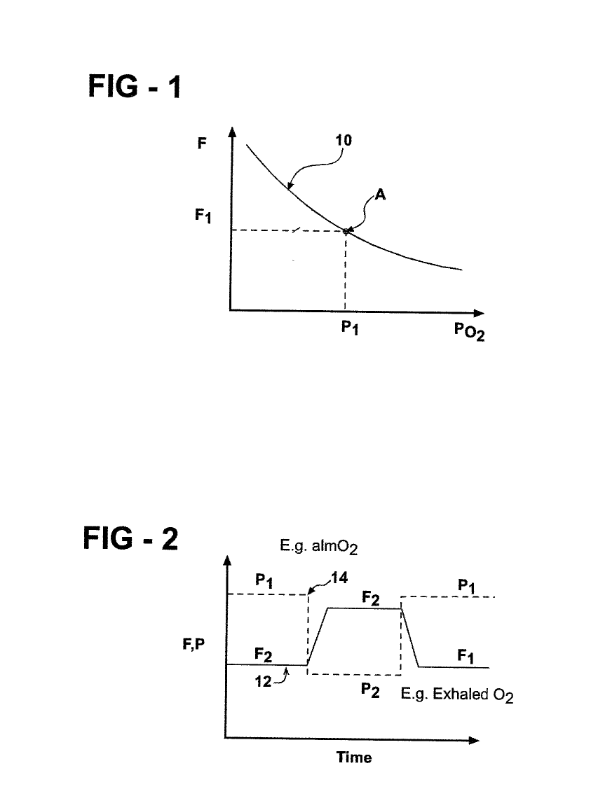

### DESCRIPTION OF THE DIAGRAMS OF FIGS. 1-5

FIG. 1 shows a fluorescence-oxygen relationship (curve 10) providing a correlation between a fluorescence intensity signal F and an oxygen partial pressure P, for a fluorescent film exposed to different partial pressures of oxygen. A similar relationship exists between the fluorescence decay time and oxygen partial pressure. If the form of the curve is known, for example, if the curve follows the Stern-Vollmer equation with a known Stem-Vollmer coefficient, the curve can be fully defined by determining a single point (e.g., point A on curve 10), which may be called single-point calibration. In respiratory analysis, point A can correspond to a known inhaled oxygen partial pressure, such as atmospheric air. Hence, point A can correspond to the sensor exposure to atmospheric air; in such case, P₁ corresponds to the atmospheric oxygen partial pressure, and F₁ to the corresponding fluorescence intensity.

A single-point calibration method can be applied for a sensor having no reference channel using the fluorescence intensity (and/or decay time) of the sensing channel exposed to inhaled gases of known oxygen partial pressure, for example atmospheric gas.

Temperature corrections can be made by determining temperatures in a number of ways including: by using a thermistor; by fluorescence decay time measurements from the sensing or reference channels; by a non-degrading fluorescent element, such as a rare earth doped glass; by thermo-optical effects or speed of sound; or by some other temperature determination method. Temperature correction methods are further described by Klimant et al. in U.S. Patent Application Serial No. 20010001642-A1, the contents of which are incorporated herein by reference. A pressure (or altitude) correction can also be made by the use of a pressure sensor.

FIG. 2 shows the fluorescence signal (curve 12) from a fluorescence-oxygen sensor exposed to gases of changing oxygen partial pressure (curve 14). The oxygen partial pressure in the gases alternates between a first known partial pressure P₁ (for example, atmospheric oxygen partial pressure), and a second unknown partial pressure P₂ (for example, an oxygen partial pressure in exhaled gases, neglecting time dependence of the exhalation composition). The unknown gas partial pressure P₂ can be determined in FIG. 2 by knowing the fluorescence-oxygen relationship of the sensor, for example the numerical values corresponding to the curve of FIG. 1 for the sensor; by the Stern-Vollmer coefficients; or by other numerical parameters to be inserted into an analytical or numerical expression of the fluorescence-oxygen relationship. However, as indicated above, an unknown degree of sensor degradation will make the curve of FIG. 1 unreliable.

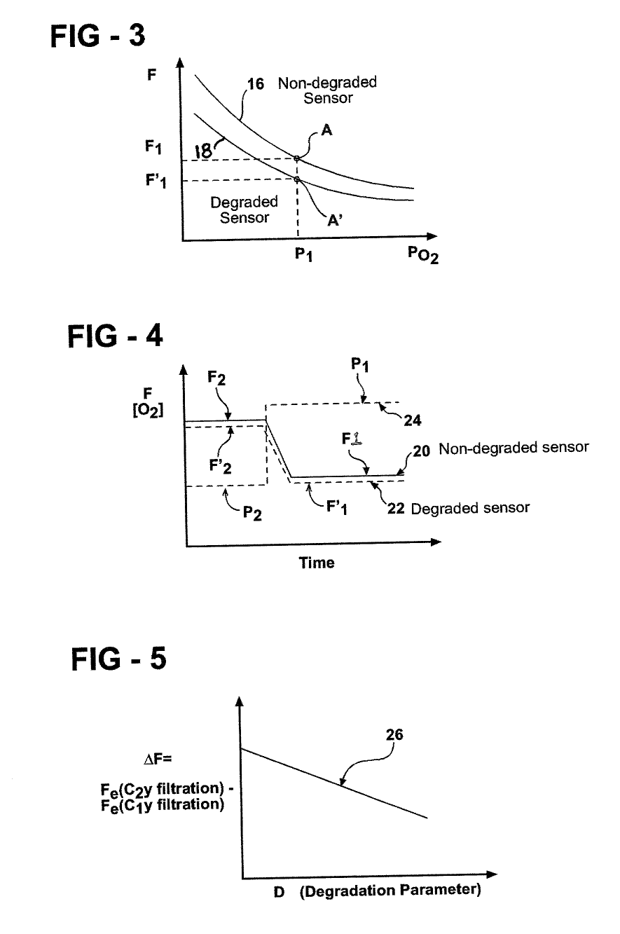

FIG. 3 shows an initial fluorescence-oxygen relationship for an non-degraded sensor (curve 16), and a modified fluorescence-oxygen relationship for a degraded sensor (curve 18). Exposure to a known partial pressure of oxygen, for example, atmospheric partial pressure, gives an initial value of fluorescence intensity F₁ (curve 16); but after degradation, a modified value of fluorescence intensity F₁′ (curve 18) is obtained.

FIG. 4 shows the fluorescence signals obtained from a non-degraded sensor (curve 20) and from a degraded sensor (curve 22) in response to different oxygen partial pressures (curve 24).

For a given exhaled oxygen partial pressure, we can define ΔF=F₂−F₁ for ΔP=P₁−P₂. However, degradation of the fluorescent film will cause ΔF to decrease for constant ΔP, changing the form of curve 10 of FIG. 1 in a manner not properly accounted for in conventional sensing systems. The value of ΔF will be a function of the degradation parameter D.

For example, the degradation parameter D can be defined as the ratio of an initial F₁ for a device, as calibrated by the manufacturer, with a determined F₁′ (for example, as illustrated in FIG. 4) made at the time of an analytical measurement, such as a respiratory test. Hence,

D=F₁′(degraded)/F₁(initial)

Other relationships can be used to defined D in term of F₁′ and F₁. F₁ (initial) can be determined for a new instrument or sensor, by passing atmospheric air over the sensor. Air can be dried or at some known humidity, and can be at a known temperature and pressure. Air temperature and sensor temperature will be different if the sensor is heated, as is the case in a present embodiment of the respiratory analyzer. F₁′ (degraded) may be determined by studying the sensor response to air exposure at different aging stages. Humidity, air temperature, pressure, and sensor temperature can also be measured and used to provide additional corrections.

Such a respiratory analyzer can also be returned to the supplier for recalibration to enable the exhaled oxygen partial pressure to be determined more accurately by taking into account the degradation parameter of the fluorescent film.

The value of ΔF is related to the Stem-Vollmer coefficients of the fluorescent material. This can be measured for a new sensor in controlled conditions during an initial calibration process. The change in Stem-Vollmer coefficients can be correlated with the degradation parameter D, or some other measure of degradation, allowing the exhaled oxygen partial pressure to be more accurately determined from F₂.

FIG. 5 shows a numerical correlation (curve 26) relating the degradation parameter D and ΔF for a given P₁−P₂, or ΔP. This curve may be determined numerically for representative films and applied for use as a modified fluorescence-oxygen relationship during respiratory analysis of exhalations.

As will be described more particularly below, the present invention automatically corrects for degradation of the sensor in the following manner. The invention:

(a) utilizes a breathing gas mixture having a known concentration of the gas component of interest (e.g. oxygen) before inhalation of the gas mixture;

(b) exposes the sensor for the gas component of interest to the breathing gas mixture during inhalation to produce an inhalation output from the sensor corresponding to the measured concentration of the gas component in the inhalation;

(c) determines, from the known concentration of the gas component in the breathing gas mixture, and the measured concentration in the inhalation output, the degradation parameter representing the instantaneous degradation condition of the sensor;

(d) exposes the sensor to the breathing gas mixture during exhalation to produce an exhalation output from the sensor corresponding to measured concentration of the gas component in the exhalation;

(e) modifies the exhalation output of the sensor according to the determined degradation parameter; and

(f) computes the change in concentration from the modified exhalation output and the measured inhalation output.

As will be further described below, the breathing gas mixture is typically atmospheric air having a known concentration of the gas component of interest, e.g., oxygen, and/or carbon dioxide. For greater accuracy, the inhaled breathing gas mixture may be analyzed to determine the instantaneous humidity concentration of the atmospheric air in order to correct the known concentration of atmospheric air according to the humidity therein.

Thus, the fraction of dry air that is oxygen, carbon dioxide, nitrogen and other gases, varies only slightly from location to location. For example, it is generally assumed to be 20.946% for oxygen, 0.033% for carbon dioxide, 78.094% for nitrogen, and 0.937% for other gases. However, the atmospheric air actually breathed is not dry air, but instead includes a portion of a water vapor which can vary according to conditions. Therefore, to provide a more accurate determination of the portion of the inhaled air which is oxygen (or carbon dioxide, etc.), the volume of the inhalation which is attributable to water vapor is preferably determined and subtracted from the measured volume of the inhalation, to provide a dry air measurement.

### DESCRIPTION OF A PREFERRED EMBODIMENT OF THE INVENTION

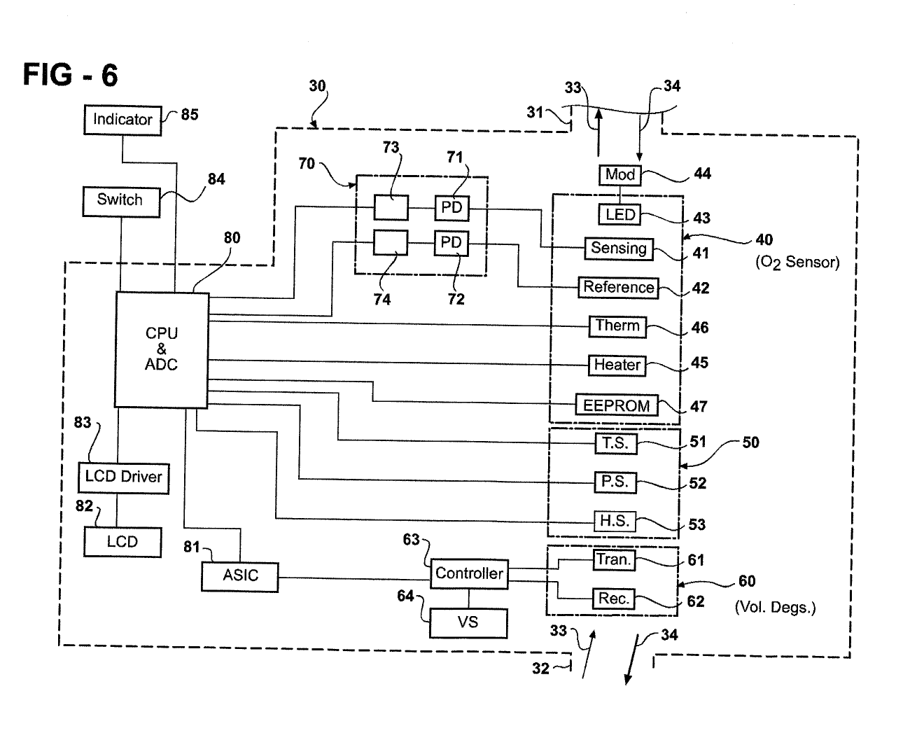

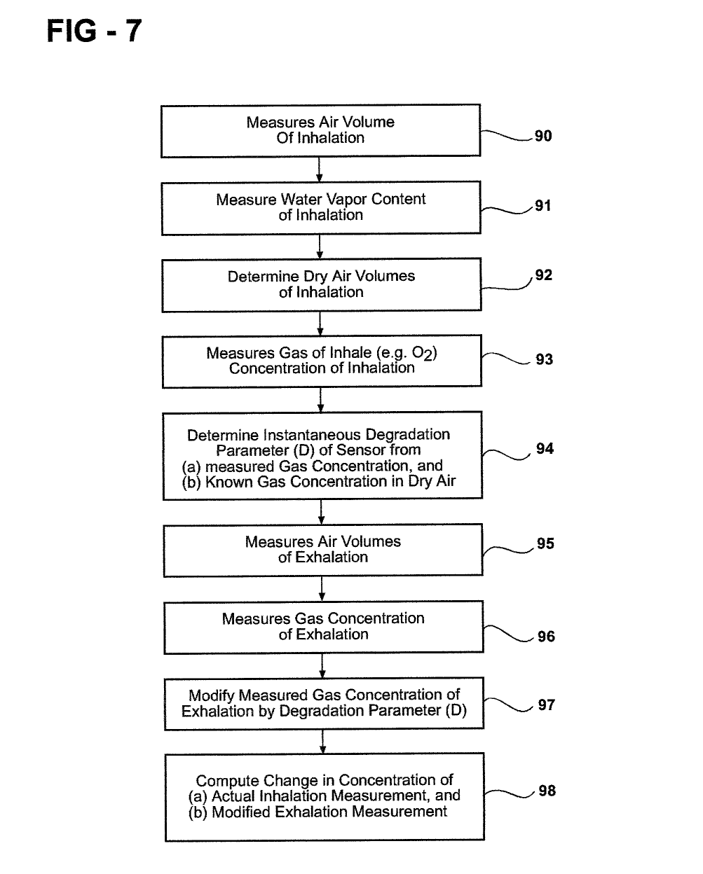

FIG. 6 is a block diagram of a respiratory analyzer constructed in accordance with the present invention, and FIG. 7 is a flow chart illustrating the operation of the respiratory analyzer of FIG. 6. The respiratory analyzer illustrated in FIG. 6 is basically of the same construction as described in the above-cited U.S. patent application Ser. No. 09/630,398, but modified in accordance with the present invention, as more particularly described in the flow chart of FIG. 7.

The Overall Construction of the Respiratory Analyzer of FIG. 6.

The respiratory analyzer illustrated in FIG. 6 is basically of the same construction as the calorimeter illustrated in FIG. 14 of the above cited U.S. patent application Ser. No. 09/630,398. It includes a housing, schematically indicated by broken-lines at 30, connectable, e.g. by a mask (not shown), to the subject's mouth and defining a common flow path for the atmospheric air during inhalations and exhalations. This common flow path includes one port 31 and another port 32. Thus, as shown in FIG. 6, during inhalations the air passes in through port 31 and out through port 32 as indicated by the arrows 33; and during exhalation, the air passes in the opposite direction as indicated by arrows 34.

Exposed to the gases flowing through the common flow channel are: a gas sensor, generally designated 40, for sensing the gas component of interest (e.g. oxygen); a humidity sensor, generally designated 50, for determining the water vapor concentration in the atmospheric air inhaled; and a flow-rate sensor, generally designated 60, for measuring the volume of the air in the respective inhalation and exhalation.

The gas component sensor 40 in this case is an oxygen sensor, although it will be appreciated that it could be or include a sensor for a different gas component, e.g. carbon dioxide, for the breathing air mixture. Oxygen sensor 40 is of the above-described fluorescent type. It includes an oxygen sensing region 41 and a reference region 42. Sensing region 41 is exposed to the air in the inhalations and exhalations so as to produce an output corresponding to the measured concentration of the oxygen therein. Reference region 42 is subjected to all the ambient conditions of sensing region 41 (e.g. temperature, light intensity, etc.), except that it is shielded from the gas flowing through the flow channel so as not to be exposed to the oxygen component thereof.

As indicated earlier, reference region of 42 is optional to increase the accuracy of the calorimeter since it compensates the output of the sensing region 41 for all ambient conditions to which the sensing region 41 is subjected to, except for degradation of the sensor during usage. This is because the reference region 42 is not subjected to the air flow through the flow channel and therefore would not degrade during usage to the same extent as the sensing region 41.

According to the present invention, compensation for degradation of the reference region 42 of the sensor 40 is provided in a different manner, as will be described more particularly below with respect to the flow chart of FIG. 7.

The oxygen sensor 40 further includes a heater 45 to warm the two fluorescent regions 42 to approximately 45° C., which temperature is monitored by a thermistor 46. The oxygen sensor 40 further includes a storage device 47 in the form of an EEPROM, containing calibration data for the oxygen sensor 40.

The humidity sensor 50 within the flow channel includes: a temperature sensor 51 for measuring the temperature of the air flowing therethrough; a pressure sensor 52 for measuring the pressure thereof; and a water vapor sensor 53 for measuring the water vapor content of the air. As briefly described earlier, humidity sensor 50 measures the instantaneous humidity concentration of the air passing through the flow channel in order to subtract, from the measured volume of the air flowing therethrough, the volume of water vapor therein and thereby to provide a more accurate dry air measurement of the oxygen content of the air.

The flow rate sensor 60 measures the flow rate of the air flowing through the flow channel during each inhalation and exhalation. This flow rate is integrated over the period of the respective inhalation and exhalation to produce a measurement of the air volume in the respective inhalation and exhalation. In the described preferred embodiment, air volume sensor 60 measures the flow rate by measuring the transient time of an acoustical pulse across the flow path of the air. Thus, it includes an ultrasonic transmitter 61 at one side of the flow path; a sonic receiver 62 spaced a distance therefrom along the flow path; a controller 63 for controlling the transmitter 61 and receiver 62; and a voltage source 64 for driving the foregoing elements of the flow rate sensor 60.

The outputs of the sensing region 41 and reference region 42 of the oxygen sensor 40 are processed by a light processor circuit 70. Thus, circuit 70 includes a light detector in the form of a photodiode 71 receiving the output from the sensing region 41 of the oxygen sensor 40, and a photodiode 72 for receiving the output of the reference region 42 of the oxygen sensor 40. Both photodiodes 71 and 72 are modulated by a modulator 44 at the same frequency as the LED 43.

It will thus be seen that the output signal from photodiode 71 is reduced in intensity (quenched) by the presence of oxygen at the sensing region 41, and thereby provides a measurement of the oxygen partial pressure in the gas flowing over that region. On the other hand, the output signal from photodiode 72 is affected by all the ambient conditions of the sensing region 41, except that, since it is shielded from the gas flowing over the sensing region, the output from photodiode 72 is not reduced in intensity by the presence of oxygen in the sensing region.

Accordingly, the output signal from photodiode 72 is independent of the oxygen content of the gas flow, and thereby serves as a reference for correcting the oxygen measurement of the photodiode 71 for all the ambient conditions to which the sensing region 41 is subjected, except for the degradation of the sensing region 41 due to previous gas flows since the reference region 42 is shielded from such gas flows.

According to the present invention, compensation for degradation of the sensor is specifically provided for in the system of FIG. 6, as described below with respect to the flow chart of FIG. 7.

The electrical system illustrated in FIG. 6 further includes a CPU (central processing unit) 80, which has an ADC (analog-to-digital converter) for receiving the outputs of the oxygen sensor 40 via the light processor circuit 70, the humidity sensor 50, and the flow rate sensor 60, via an application specific integrated circuit (ASIC) 81. Alternatively, the CPU 80 could itself perform the necessary analog-to-digital conversion.

CPU 80 computes, from the foregoing inputs, the flow volumes, the oxygen concentration, the rate of consumption of oxygen, and the subject's metabolic rate. This is displayed in an LCD (liquid crystal display) 82 controlled by an LCD driver 83.

The system illustrated in FIG. 6 further includes a switch 84 for turning on the apparatus, and a light indicator 85 for indicating the condition of the apparatus.

As indicated earlier, the system illustrated in FIG. 6, except for the manner of compensating the sensor output signals for the degradation state of the oxygen sensor 40, is of substantially the same construction as described in the above-cited U.S. patent application Ser. No. 09/630,398, incorporated herein by reference; and therefore further details of the construction and operation of such a system are available from the description in that application.

Compensation for Degradation of the Oxygen Sensor.

As indicated earlier, compensation for degradation of the oxygen sensor 40 is automatically and continuously effected by determining the degradation state or parameter of the sensor during each inhalation, and using the determined degradation parameter for correcting the sensor output during the subsequent exhalation. Since the gas composition of dry atmospheric air is generally the same at all locations, the degradation parameter can thus be determined by using the known gas concentration in dry air and the measured gas concentration during the inhalation.

Operation

The flow chart of FIG. 7 illustrates the overall operation of the system for measuring changes in the concentration of a particular gas component, such as oxygen, of a breathing gas mixture after an inhalation and exhalation, and particularly the manner in which the degradation parameter of the oxygen sensor (40) is automatically determined and used for providing an accurate computation in the change of concentration of oxygen between an inhalation and exhalation.

Thus, as shown by block 90 in FIG. 7, the air volume of an inhalation is measured. This is done by utilizing the flow rate sensor 60 to measure the rate of flow during the inhalation, and then integrating the flow rate over the period of time of the inhalation.

The water vapor content of the inhalation is measured (block 91) by measuring the temperature of the inhalation by temperature sensor 51, measuring the pressure of the inhalation by pressure sensor 52, and measuring the humidity of the inhalation by water vapor sensor 53.

The dry air volume of the inhalation is then determined (block 92) by subtracting the measured water vapor content (block 91) from the measured air volume (block 90).

The instantaneous degradation parameter of the oxygen sensor 40 is then determined (block 94) from (a) the measured gas concentration and (b) the known gas concentration in dry air. Since the known value (b) represents no degradation (i.e. 100% effectiveness of the oxygen sensor), the instantaneous degradation parameter at any particular condition of the oxygen sensor may be determined by dividing the measured value (a) by the known value (b).

The air volume of the exhalation is then measured (block 95) in the same manner as the air volume of the inhalation was measured (block 90); and the gas (e.g. oxygen) concentration of the exhalation is measured (block 96) in the same manner as during the inhalation (block 91).

The measured gas concentration of exhalation is then modified by the degradation parameter determined in block 94 (block 97). Finally, the change in concentration of the gas during the inhalation and exhalation is computed from the actual inhalation measurement produced in block 93 and the modified exhalation measurement produced in block 97. For example, if the gas component of interest is oxygen, the oxygen concentration in the exhalation would be less than in the inhalation, and therefore the modified exhalation measurement of (b) above would be subtracted from the actual inhalation measurement of (a) above. On the other hand, if the gas component of interest is carbon dioxide, its concentration in the exhalation would be larger than in the inhalation, and therefore the actual inhalation measurement of (a) above would be subtracted from the modified exhalation measurement of (b) above.

While the invention has been described with respect to one preferred embodiment, it will be appreciated that this is set forth merely for purposes of example, and that many variations may be made. For example, the reference channel coupled to the reference region 42 of the sensor 40 could be omitted in order to reduce costs and/or improve compactness, or could be replaced by another sensor channel for sensing another gas component of the breathed air. Other gas component sensors could be used. In addition, fiber optical sensors could be used in order to enable illumination from a remote source and/or detection by a remote detector. Further, a flow rate measuring system other than the acoustical type described above could be used for measuring the flow rate, and thereby the flow volume.

In addition, the described method and apparatus could be used for other purposes than determining oxygen consumption and/or metabolic rates, or for measuring changes in concentration of other fluids in the breathing air mixture, including nitrogen, nitric oxide (NO), hydrogen, methane, ammonia, carbon monoxide, alkanes, such as pentane, acetone, acetaldehyde, ammonia, isoprene, isobutyric acid, n-butyric acid, isovaleric acid, n-valeric acid, propionic acid, amines (such as dimethylamine and trimethylamine), other volatile organic components, drug metabolites, respiratory indicators of drug use, bacterial metabolites, and other respiratory components. Breath components can be detected for health diagnostic purposes, such as for the diagnosis of cancer, precancer, diabetes and other metabolic disorders, or for detecting fat burning, liver or kidney malfunction, lung disorders, respiratory tract disorders, oral disorders, infections, and other disorders. Breath components can also be detected for lifestyle diagnosis, for example to determine drug or alcohol use, or for the detection of pathogens, such as bacteria and viruses using various immunological methods.

In addition, the methods and apparatus described herein can also be applied to the design of respiratory analyzers for use only to study exhalations, such as certain conventional spirometers. For example, the gas sensor response to atmospheric air, either during an inhalation test, or prior to a test, can be used to determine sensor degradation effects. If the gas component of interest is not present in the atmosphere, atmospheric exposure can be used to zero the sensor response.

While the invention has been described above primarily with respect to one preferred embodiment, it will be appreciated that this is set forth merely for purposes of example, and that many other variations, modifications and applications of the invention may be made.

What is claimed:

1. A method of measuring changes in the concentration of a particular gas component of a breathing gas mixture after inhalation and exhalation of said breathing gas mixture, which breathing gas mixture has a known concentration of said gas component before inhalation thereof, said method comprising:
exposing a sensor for said gas component to said breathing gas mixture during inhalation to produce an inhalation output from said sensor corresponding to the measured concentration of said gas component in said inhalation, said sensor being degradable by usage;
determining, from said known concentration of said gas component in the breathing gas mixture and said measured concentration in said inhalation output, a degradation parameter representing the instantaneous degradation condition of said sensor;
exposing said sensor to said breathing gas mixture during exhalation to produce an exhalation output from said sensor corresponding to the measured concentration of said gas component in said exhalation;
modifying said exhalation output of the sensor according to said determined degradation parameter;
and computing said change in concentration from said modified exhalation output and said measured inhalation output.

2. The method according to claim 1, wherein said gas component is oxygen, and said change in concentration thereof is computed by subtracting said modified exhalation output from said measured inhalation output.

3. The method according to claim 1, wherein said gas component is carbon dioxide, and said change in concentration thereof is computed by subtracting said measured inhalation output from said modified exhalation output.

4. The method according to claim 1, wherein said degradable sensor is a fluorescent-type sensor.

5. The method according to claim 1, wherein said degradable sensor produces an output corresponding to the partial pressure of said gas component.

6. The method according to claim 1, where said breathing gas mixture is atmospheric air.

7. The method according to claim 6, where the instantaneous humidity concentration of said atmospheric air is determined and used to correct the known concentration of said gas component in the atmospheric air for determining said degradation parameter.

8. The method according to claim 1, wherein a reference sensor for said gas component, while exposed to the instantaneous temperature as said first-mentioned sensor but not exposed to said breathing gas mixture, is utilized during the inhalation and exhalation to produce reference outputs corresponding to said instantaneous temperature, which reference outputs are used to correct the inhalation and exhalation outputs of said first-mentioned sensor for the instantaneous temperature during said inhalation and exhalation.

9. A method of measuring the metabolic rate of a subject, comprising:
measuring, in accordance with claim 1, changes in the concentration of a particular gas component of a breathing gas mixture after inhalation and exhalation by said subject;
measuring the volume of said breathing gas mixture inhaled and exhaled by said subject;
and multiplying said measured volume by said measured changes in the concentration of said gas component to provide a measurement of said metabolic rate of the subject.

10. The method according to claim 9, wherein said measured changes in concentration represent the oxygen in said breathing gas mixture consumed by the subject.

11. The method according to claim 9, wherein said measured changes in the concentration represent the carbon dioxide added to said breathing gas mixture by the subject.

12. The method according to claim 9, wherein said volume is measured by measuring the flow rate of said breathing gas mixture during said inhalation and exhalation, and integrating said measured flow rate over the time interval of said inhalation and exhalation.

13. The method according to claim 12, wherein said flow rate is measured by measuring the transient time of an acoustical pulse across a flow path of said breathing gas mixture.

14. The method according to claim 13, wherein said breathing gas mixture is atmospheric air.

15. The method according to claim 14, where the instantaneous humidity concentration of said atmospheric air is determined and used to correct the known concentration of said gas component in the atmospheric air for determining said degradation parameter.

16. The method according to claim 9, wherein a reference sensor for said gas component, while exposed to the instantaneous temperature as said first-mentioned sensor but not to exposed said breathing gas mixture, is utilized during the inhalation and exhalation to produce reference outputs corresponding to said instantaneous temperature, which reference outputs are used to correct the inhalation and exhalation outputs of said first-mentioned sensor for the instantaneous temperature during said inhalation and exhalation.

17. A respiratory analyzer for measuring changes in the concentration of a particular gas component of a breathing gas mixture after inhalation and exhalation of the breathing gas mixture, which breathing gas mixture has a known concentration of said gas component before inhalation thereof; said analyzer comprising:
a common flow path for said breathing gas mixture during inhalations and exhalations thereof;
a sensor exposed to said breathing gas mixture in said flow path to produce an inhalation output from the sensor and an exhalation output from the sensor corresponding to the measured concentration of said gas component in said inhalations and exhalations, respectively;
and a processor for:
determining, from said known concentration of said gas component in the breathing gas mixture and said measured concentration in said inhalation output from the sensor, a degradation parameter representing the instantaneous degradation condition of said sensor;
modifying said exhalation output of the sensor according to said determined degradation parameter;
and computing said change in concentration from said modified exhalation output and said measured inhalation output from the sensor.

18. The analyzer according to claim 17, wherein:
said sensor measures the concentration of oxygen in said inhalations and exhalations;
and said processor computes said change in concentration of the oxygen by subtracting said modified exhalation output from said measured inhalation output of the sensor.

19. The analyzer according to claim 17, wherein:
said sensor measures the concentration of carbon dioxide in said inhalations and exhalations;
and said processor computes said change in concentration of the carbon dioxide by subtracting said measured inhalation output from said modified exhalation output of the sensor.

20. The analyzer according to claim 17, wherein said degradable sensor is a fluorescent-type sensor.

21. The analyzer according to claim 17, wherein said degradable sensor produces an output corresponding to the partial pressure of said gas component.

22. The analyzer according to claim 21, wherein said breathable gas mixture is atmospheric air, and said analyzer determines the instantaneous humidity concentration of said atmospheric air and uses same to correct the known concentration of said gas component in the atmospheric air for determining said degradation parameter.

23. The analyzer according to claim 17, wherein said analyzer further comprises a reference sensor for said gas component, which reference sensor, while exposed to the instantaneous temperature as said first-mentioned sensor but not exposed to said breathing gas mixture, is utilized during the inhalation and exhalation to produce reference outputs corresponding to said instantaneous temperature, which reference outputs are used to correct the inhalation and exhalation outputs of said first-mentioned sensor for the instantaneous temperature during said inhalation and exhalation.

24. An indirect calorimeter for measuring the metabolic rate of a subject, comprising:
a respiratory analyzer according to claim 17 for measuring changes in the concentration of a particular gas component of a breathing gas mixture after inhalation and exhalation thereof;
and an apparatus for measuring the volume of said breathing gas mixture inhaled and exhaled by the subject;
said processor also multiplying said measured volume by said measured changes in the concentration of said gas component to provide a measurement of the metabolic rate of the subject.

25. The calorimeter according to claim 24, wherein said sensor measures the oxygen component and thereby the oxygen consumption between said inhalations and exhalations.

26. The calorimeter according to claim 24, wherein said sensor measures the carbon dioxide, and thereby the carbon dioxide addition between said inhalations and exhalations.

27. The calorimeter according to claim 24, wherein said apparatus for measuring the volume of the gas component measures the flow rate of said breathing gas mixture during said inhalations and exhalations, and integrates said measured flow rates over the time intervals of said inhalations and exhalations.

28. The calorimeter according to claim 24, wherein said apparatus measures said flow rate by measuring the transient time of an acoustical pulse across a flow path of said breathing gas mixture.

29. The calorimeter according to claim 24, wherein said calorimeter further comprises apparatus for determining the instantaneous humidity concentration of said atmospheric air, and for using same to correct the known concentration of said gas component in the atmospheric air for determining said degradation parameter.

30. The calorimeter according to claim 24, wherein said calorimeter further comprises a reference sensor for said gas component; said reference sensor being exposed to the instantaneous temperature as said first-mentioned sensor but not to said breathing gas mixture; said reference sensor being utilized, during an inhalation and an exhalation, to produce reference outputs corresponding to said instantaneous temperature; said reference outputs being used to correct the inhalation and exhalation outputs of said first-mentioned sensor for the instantaneous temperature during said inhalation and exhalation.
